(19) United States
(12) Patent Application Publication
(10) Pub. No.: US 2010/0255478 A1
(43) Pub. Date: Oct. 7, 2010

(54) TARGETS FOR USE IN DIAGNOSIS, PROGNOSIS AND THERAPY OF CANCER

(76) Inventor: Charis Eng, Cleveland Heights, OH (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/727,039

(22) Filed: Mar. 18, 2010

Related U.S. Application Data
(60) Provisional application No. 61/161,254, filed on Mar. 18, 2009.

(51) Int. Cl. C12Q 1/68

(52) U.S. Cl. 435/6

(57) ABSTRACT

The invention is directed to a method of diagnosing cancer, or susceptibility to cancer, in an individual in need thereof comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity at the one or more loci is indicative of a diagnosis of cancer, or susceptibility to cancer, in the individual. Also provided herein are kits for use in diagnosing cancer or susceptibility to cancer in an individual comprising one or more regents for detecting the presence of a homozygosity at one or more loci selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477 and instructions for use.

### RELATED APPLICATIONS

This application claims the benefit of U.S. Provisional Application No. 61/161,254, filed on Mar. 18, 2009 and is related to U.S. Provisional Application No. 61/070,089, filed on Mar. 20, 2008. The entire teachings of the above application(s) are incorporated herein by reference.

### GOVERNMENT SUPPORT

The invention was supported, in whole or in part, by grants 1P01CA97189-01A2 and 1P50/U54CA113001-01 from the National Cancer Institute, and DAMD-02-1-0118 from the Department of Defense Prostate Cancer Research Program. The Government has certain rights in the invention.

### BACKGROUND OF THE INVENTION

Cancer is a multigenic disease resulting from both germline susceptibility and somatic events. A greater understanding of cancer at the etiologic level is needed to improve diagnosis and therapy of this disease.

### SUMMARY OF THE INVENTION

As described herein, while studying loss of heterozygosity (LOH) in cancer tissues, a low frequency of heterozygosity in cancer patients compared with controls was observed, raising the question whether homozygosity could play a role in cancer predisposition. The frequency of germline homozygosity in a large series of patients with 3 different types of solid tumors compared with population-based controls was determined.

Germline and corresponding tumor DNA isolated from 385 patients with carcinomas (147 breast, 116 prostate, and 122 head and neck carcinomas) were subjected to whole genome (345-microsatellite marker) LOH analysis. The main outcome measures were frequency of homozygosity at microsatellite markers in cancer cases versus controls and frequency of somatic LOH in cancers at loci with the highest homozygosity. Sixteen (16) loci in common among the 3 cancer types were identified, with significantly increased germline homozygosity frequencies in the cancer patients compared with controls (P<0.001). In the cases who happened to be germline heterozygous at these 16 loci, a mean (SD) LOH frequency of 58% (4.2%) compared with 50% (7.5%) at 197 markers without increased germline homozygosity (P<0.001) was found. Across the genome, this relationship holds as well (r=0.46; 95% confidence interval, 0.37-0.53; P<0.001). The association of specific loci with high germline homozygosity frequencies was validated in an independent, single-nucleotide polymorphism-based, public data set of 205 lung carcinomas from white individuals (P<0.05 to P<0.001) as well as the correlation between genome-wide germline homozygosity and LOH frequencies (r=0.21; 95% confidence interval, 0.18-0.24; P<0.001). In a study of 4 different types of solid tumors (the data for 3 types validated in a fourth type), increased germline homozygosity occurred at specific loci. When the germline was heterozygous at these loci, high frequencies of LOH/allelic imbalance occurred at these loci in the corresponding carcinomas (see Assie, G, et al., JAMA 2008; 299(12):1437-1445, which is herein incorporated by reference in its entirety).

Accordingly, in one aspect, the invention is directed to a method of diagnosing cancer in an individual in need thereof comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity at the one or more loci is indicative of a diagnosis of cancer in the individual.

In another aspect, the invention is directed to a method of diagnosing susceptibility to cancer in an individual in need thereof comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity at the one or more loci is indicative of a diagnosis of susceptibility to cancer in the individual.

Also provided herein are kits for use in diagnosing cancer or susceptibility to cancer in an individual comprising one or more regents for detecting the presence of a homozygosity at one or more loci selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477 and instructions for use.

### BRIEF DESCRIPTION OF THE DRAWINGS

FIG. 1 is a schematic representation of the Knudson 2-Hit Hypothesis. A germline mutation in a tumor suppressor gene is inherited (first hit) on the red chromosome (A). The paired white chromosome is wild-type (normal). A marker near the mutation spot that is heterozygous in the germline is shown at the top. When the wild-type (white) chromosome is lost during tumorigenesis, the loss-of-heterozygosity (LOH) scan shows only 1 remaining peak (B).

FIG. 2 illustrates the loss-of heterozygosity analysis in tumors. (A) Illustration of germline heterozygosity (2 peaks on loss-of-heterozygosity ([LOH] scan) and homozygosity (single peak on LOH scan) (left panel). When the germline is heterozygous, the tumor can retain both alleles and is said to have retention of heterozygosity (ROH) (top right panel), or lose 1 allele and undergo (LOH (middle and bottom right panel). (B) Four examples from genotyping output from 2 patient samples. Each of the top or bottom panel shows the genotyping readout from germline DNA (top) and somatic tumor DNA (bottom). The bottom vertical pairs are enlargements of the top panel genotyping readout, which show microsatellite markers that have retained (R) heterozygosity (ATA5A09, bottom left) and that have undergone loss of heterozygosity (L, D5S1462 and D3S1763, bottom-right 2 panels).Marker D8S1179 is homozygous in the germline (H). Note that because the tumor cells were procured by laser capture microdissection, virtually all scoring of LOH and retention of heterozygosity are very clear.

FIG. 3 shows the mapping of germline homozygosity frequencies in cancer patients. For each microsatellite marker, the germline homozygosity frequency in 3 cancer types (head and neck squamous cell carcinoma, prostate carcinoma, and breast carcinoma) was compared with the reference homozygosity frequency. A binomial test was used and P values were adjusted by false discovery rate. The −log₁₀ P value of the comparison is plotted vs the physical position, with the 22 autosomes represented. The dashed line represents the 0.05 significance limit. Loci with increased homozygosity common to all 3 cancer types are also shown.

FIG. 4 shows the frequency of germline homozygosity of microsatellite markers in all cancer. Frequency of germline homozygosity of microsatellite markers among all cancer patients as a function of the frequency of loss of heterozygosity/allelic imbalance (LOH/AI) in tumors whose corresponding germline is heterozygous at the same loci, showing a correlation coefficient of 0.46 (Perason r, P<0.001).

### DETAILED DESCRIPTION OF THE INVENTION

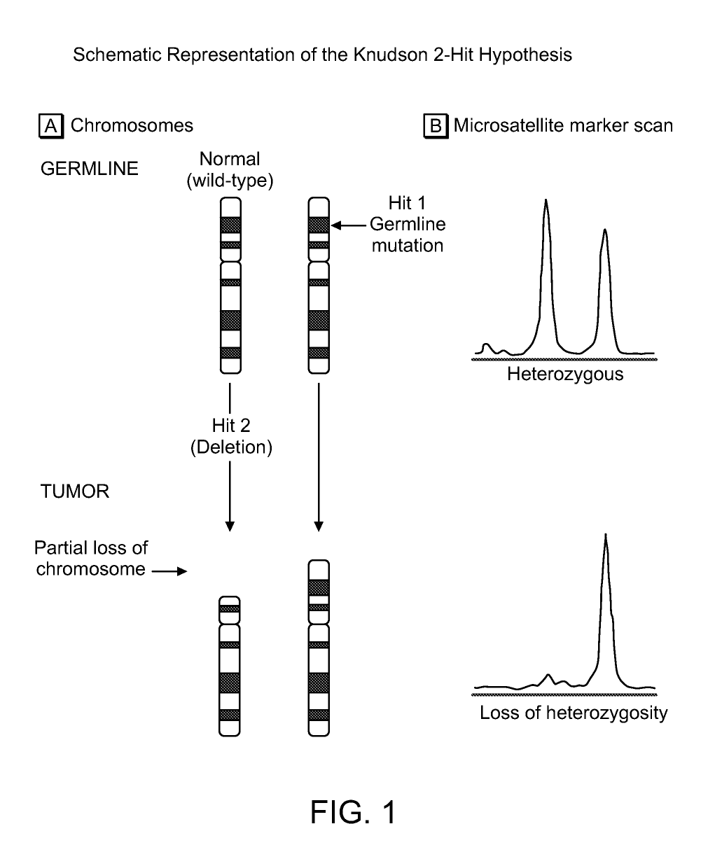

Knudson first introduced the concept of both a germ-line predisposition and a somatic event for the inactivation of tumor suppressors in cancer with the “2 hit hypothesis” (Knudson A G Jr. Proc. Natl. Acad. Sci. USA. 1971; 68(4):820-823). This theory applies to heritable cancer syndromes caused by mutations in tumor suppressors with high penetrance. The first hit is an inherited mutation (mutant allele) in the germline while the most simple second hit is somatic loss of the wild-type (normal) allele, leaving a single copy of the mutant allele (FIG. 1). More commonly, the second hit is a combination of multiple and complex somatic events (Balmain A, Gray 1, Ponder B. Nat. Genet. 2003; 33 (suppl):238-244). In contrast, most sporadic cancers are complex traits wherein the germline predisposition is likely related to several low penetrance genomic variants, present in either oncogenes or tumor suppressor genes.

As described herein, while studying loss of heterozygosity/allelic imbalance (LOH/AI) in cancer tissues using microsatellite markers in germline and in corresponding tumors (Fukino K. et al. Cancer Res. 2004; 64(20):17237-7236; Fukino K, et al. JAMA. 2007; 297(19):2103-2111; Kurose K, et al., Hum. Mol. Genet. 2001; 10(18)1907-1913; Weber F. et al., JAMA 2007; 297(2):187-195; Sarquis M S, et al., J Endocrinol. Metab. 2006; 91(1):262-269), a low frequency of germline heterozygosity in cancer patients compared with controls was observed (C. E., unpublished data, 1993-2006), raising the question whether homozygosity could play a role in cancer predisposition. Further evidence suggesting a role of homozygosity in cancer is brought by studies that showed an increased risk of cancer in inbred populations (Rudan I, et al., J Med Genet. 2003; 40(12):925-932; Sherri S A, et al., Lancet 1991; 338(8772): 954; Rudan I. Hum Bio 1999; 71(2):173-187; Simpson I L, et al., Am J. Obstet Gynecol. 1981; 141 (6):629-636; Bhattacharya P, et al., Cancer Lett 2002; 188(1-2):207-211) by several reports identifying homozygous loci associated with cancer (Bhattacharya P, et al., Cancer Lett 2002; 188(1-2):207-211; Deligezer U, et al. In Vivo 2005; 19(5):889-893) and from experimental animal inbreeding (e.g., backcrossing mice) increasing tumor incidence. Specific situations of homozygosity have also been directly associated with cancer, such as uniparental isodisomy through altered imprinting (Henry I, et al. Nature 1991; 351(6328): 665-667).

Homozygosity is common in humans and extended homozygote tracts have been described in several studies (Broman K W, et al., Am. J. Hum. Genet. 1999; 65(6):1493-1500; Clark A G, Am. J. Hum. Genet. 1999; 65(6):1489-1492; Gibson, J. et al., Hum. Mol. Genet. 2006; 15(5):789-795; Simon-Sanchez J. et al. Hum. Mol. Genet. 2007; 16(1):1-14). Cancer susceptibility genes are also numerous in the genome. As described herein, these facts together increase the likelihood that homozygosity occurs in the loci of cancer susceptibility genes. Indeed, shown herein is that germline homozygosity at these loci somehow contribute to cancer predisposition. In the case of homozygous tumor suppressor genes, recessive mutant alleles would result in an altered tumor suppressor function with no need of any additional somatic event. In the case of dominant alleles in an oncogene, homozygosity could result in a superoncogenic effect.

Therefore, it was hypothesized that germline homozygosity lends low penetrance susceptibility to cancer by the potential mechanism of affecting the function of numerous genes throughout the genome. Described herein is how this hypothesis was systematically addressed by looking at series of patients with 3 different types of solid tumors for genomic regions with increased germline homozygosity compared with controls and by exploring somatic LOH in the cancers of patients who happened to be heterozygous at these loci.

The data described herein derived from 3 different solid tumors, validated in a fourth, demonstrate that high frequencies of germline homozygosity at specific markers (loci) are associated with these cancers compared with controls and that at the genome-wide level, there is a direct relationship between frequencies of germline homozygosity and somatic LOH if the germline is heterozygous.

Accordingly, provided herein are methods of diagnosing cancer (e.g., breast, prostrate, head and neck carcinomas) or susceptibility to cancer in an individual in need thereof comprising detecting homozygosity at one or more loci of the individual's nucleic acid.

Heterozygosity denotes the presence of two alleles which can be individually discriminated by slight, minor differences in DNA sequence commonly found at microsatellites, which are segments of DNA composed of variable numbers of short repeat units that occur in predictable locations within the genome but vary in absolute length according to the number of repeats. Microsatellite markers can be used to evaluate the two different copies or alleles of the human genome. In the normal state, the two alleles can be distinguished from a each other and are said to exist in a state of heterozygosity. When mutations are acquired which typically involve deletion of all or part of an allele, one of the two copies is lost from the cell by deletion leading to a loss of heterozygosity.

“Loss of heterozygosity/alleleic imbalance” typically refers to the loss of a portion of a chromosome in somatic cells (e.g., a deletion, mutation, or loss of an entire chromosome (or a region of the chromosome) from the cell nucleus). Since only one of the two copies of the affected chromosomal region originally present in an individual's genome will remain in cells which have undergone LOH, all polymorphic markers within the region will be or appear to be “homozygous”; i.e., these cells will have lost heterozygosity for these markers. Comparison of marker genotypes in a population of cells that are suspected of having undergone LOH with genotypes of normal tissue from the same individual allows for the identification of LOH or homozygosity, and for mapping the extent of the loss.

Examples of “loci” that when homozygous are “markers” of cancer or susceptibility to cancer in an individual include D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477. Therefore, in a particular aspect, the invention is directed to methods of diagnosing cancer or susceptibility to cancer in an individual comprising detecting the presence of homozygosity at one or more loci selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477 in the individual, wherein homozygosity at the one or more of sixteen (16) specific loci in the individual is indicative of a diagnosis of cancer or susceptibility to cancer in the individual. In particular aspects, homozygosity occurs (e.g., a higher frequency of homozygosity occurs, for example, compared to a control) in one, two, three, four, five, six, seven, eight, nine, ten, eleven, twelve, thirteen, fouteen, fifteen, or sixteen of the loci. In one aspect, homozygosity occurs (e.g., a higher frequency of homozygosity occurs, for example, compared to a control) in all sixteen loci.

As shown herein, homozygosity at the one or more loci can occur in the individual's germline nucleic acid, referred to herein as germline homozygosity. Such homozygosity can be detected, for example, in genomic DNA of the individual. In addition, or alternatively, homozygosity at the one or more loci can occur in the individual's somatic nucleic acid, referred to herein as somatic homozygosity. Such homozygosity can be detected, for example, in tumor DNA of the individual.

As described herein the methods can be used to detect or diagnose cancer or susceptibility to cancer. Examples of the types of cancers which can be detected or diagnosed include breast cancer, prostate cancer, head and neck squamous cell carcinoma, lung cancer, colorectal cancer, pancreatic cancer, bladder cancer and the like.

The methods provided herein can further comprise obtaining a sample comprising the individual's nucleic acid, such as DNA (e.g., genomic DNA), RNA, and the like, from the individual. The sample obtained can be from a non-malignant source (e.g., an individual's normal or non-cancerous tissue), a malignant source (e.g., an individual's cancerous tissue), or a combination thereof. Suitable samples include a tissue sample (e.g., organ tissue such as skin, muscle, lung; placental tissue), a cell sample (e.g., fetal cells, tumor cells), a fluid sample (e.g., blood (peripheral blood cells such as peripheral leukocytes), a tumor sample (e.g., epithelial tissue or stromal tissue from a tumor), amniotic fluid, cerebrospinal fluid, urine, lymph) and any combination thereof. In addition, as used herein a cell can be a germ cell or somatic cell. Suitable cells can be of, for example, mammalian (e.g., human) origin. In particular embodiments, the sample used to detect homozygosity at the one or more of the loci is from normal tissue. In another embodiment, the sample used to detect homozygosity at the one or more of the loci is from carcinomatous epithelium of a tumor.

Methods of obtaining such samples and/or extracting nucleic acid from such samples are described herein and known to those of skill in the art. In a particular embodiment, homozygosity at one or more specific loci can be detected in a sample (e.g., tissue, cell, fluid) from the tumor epithelium and/or the surrounding stroma of the tumor epithelium in the individual. The tumor epithelium and/or surrounding stroma can be obtained using any suitable method known in the art such as laser capture microdissection (LCM). In addition, the DNA can be extracted and amplified (e.g., using a polymerase chain reaction (PCR) method), and the LOH/AI at one or more specific loci can be detected, using any suitable methods known in the art, as described herein. As will be apparent to one of skill in the art, methods other than those described herein can be used.

Suitable methods for detecting homozygosity at the one or more loci of the individual's nucleic acid are also described herein. For example homozygosity at the one or more loci of the individual's nucleic acid can be detected using polyermase chain reaction to amplify the individual's nucleic acid thereby producing amplified nucleic acid, and genotyping the amplified nucleic acid. As is also apparent to one of skill in the art, methods other than those described herein can be used.

The methods described herein can further comprise comparing the homozygosity at the one or more loci of the individual's nucleic acid to the homozygosity at the one or more loci of a control. In one aspect, an increase in frequency of the homozygosity at the one or more loci of the individual's nucleic acid (occurs at a higher frequency) compared to frequency of the homozygosity at the one or more loci of the control, is indicative of a diagnosis or cancer in the individual or susceptibility to cancer in the individual.

Suitable controls for use in the methods provided herein are apparent to those of skill in the art. For example, a suitable control can be established by assaying one or more (e.g., a large sample of) samples from one or more individuals in which homozygosity is absent or occurs at a low frequency at the one or more the loci described herein. Examples of such controls include one or more samples from a normal (non-malignant) individual (a sample from one or more individuals that do not have cancer or are not susceptible to cancer). Alternatively, a control can be obtained using a statistical model to obtain a control value (standard value; known standard). See, for example, models described in Knapp, R. G. and Miller M. C. (1992) Clinical Epidemiology and Biostatistics, William and Wilkins, Harual Publishing Co. Malvern, Pa., which is incorporated herein by reference.

As used herein the term “individual” includes animals such as mammals, as well as other animals, vertebrate and invertebrate (e.g., birds, fish, reptiles, insects (e.g., Drosophila species), mollusks (e.g., Aplysia). Preferably, the animal is a mammal. The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include primates (e.g., humans, monkeys, chimpanzees), rodents (e.g., rats, mice, guinea pigs) and ruminents (e.g., cows, pigs, horses).

Identification of the loci or markers of cancer described herein provide for methods of detecting (diagnosing) cancer, or susceptibility to cancer in an individual in need thereof. Therefore, in one aspect, the invention provides methods of screening an asymptomatic individual for cancer comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity at the one or more loci is indicative of a diagnosis of cancer in the asymptomatic individual. Such individuals can be monitored at regular intervals (e.g., once every 6 months; once a year; once every two years, five years, ten years).

In another aspect, the invention provides methods of detecting recurrence of cancer in an individual that is in remission, or has been treated for the cancer comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity at the one or more loci is indicative of a recurrence of cancer in the individual (e.g., there is no change in the frequency of homozygosity; the frequency of homozygosity has increased; the frequency of homozygosity is higher compared to the homozygosity of a suitable control).

In another aspect, the invention provides methods of monitoring a treatment regimen for cancer in an individual comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity (e.g., there is no change in the frequency of homozygosity after treatment; the frequency of homozygosity has increased after treatment; the frequency of homozygosity is higher compared to the homozygosity of a suitable control) at the one or more loci is indicative that the treatment regimen is unsuccessful. Alternatively, if homozygosity is less frequent or absent after treatment (e.g., or the same as, absent, or less than the frequency of homozygosity of a suitable control) at the one or more loci this indicates that the treatment regimen is successful.

Also provided herein are kits for use in diagnosing cancer or susceptibility to cancer in an individual comprising one or more regents for detecting homozygosity at one or more loci selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477. For example, the kit can comprise hybridization probes, restriction enzymes (e.g., for RFLP analysis), allele-specific oligonucleotides, and antibodies. In a particular embodiment, the kit comprises at least contiguous nucleotide sequence that is substantially or completely complementary to a region of one or more of the loci comprising the homozygosity. For example, the nucleic acids can comprise at least one sequence (contiguous sequence) which is complementary (completely, partially) to one or more loci comprising homozygosity that is associated with cancer. In one embodiment, the one or more reagents in the kit are labeled, and thus, the kits can further comprise agents capable of detecting the label. The kit can further comprise instructions for detecting cancer using the components of the kit.

### Experimentation

### Methods

### Samples and Genomic DNA Preparation

Germline and tumor (somatic) genomic DNA was isolated using standard protocols from 385 patients with 3 types of invasive carcinomas: 147 breast, 116 prostate and 122 head and neck squamous cell carcinomas. Patients with widely metastatic disease (TXNXM1) were excluded from the study. Tumor DNA was extracted specifically from the carcinomatous epithelium using laser capture microdissection, as previously described (Fukino K. et al. Cancer Res. 2004; 64(20):17237-7236; Fukino K, et al. JAMA. 2007; 297(19):2103-2111). In addition, the corresponding germline DNA for each patient was procured either from peripheral blood (n=32) or from normal tissue using a different tissue block containing only normal tissue (n=353). The use of normal tissue as a reliable source of germline DNA was validated previously (Fukino K. et al. Cancer Res. 2004; 64(20):17237-7236; Fukino K, et al. JAMA. 2007; 297(19):2103-2111; Weber F. et al., JAMA 2007; 297(2):187-195; Patocs A, et al. N. Eng. J. Med. 2007; 357(25): 2543-2551).

The patients were of northern and predominantly western European ancestry by self-report. Hence, controls were chosen for matched ancestry, which is important for polymorphic marker frequency analysis. The head and neck squamous cell carcinoma and breast cancer series have been previously described in detail. (Fukino K. et al. Cancer Res. 2004; 64(20):17237-7236; Fukino K, et al. JAMA. 2007; 297(19):2103-2111; Weber F. et al., JAMA 2007; 297(2):187-195; Patocs A, et al. N. Eng. J. Med. 2007; 357(25): 2543-2551). The study, which used anonymized unlinked samples, was approved under exempt status by the Cleveland Clinic institutional review board for human subjects protection.

### Genome-Wide Microsatellite Analysis

Polymerase chain reaction was performed as previously described. (Fukino K. et al. Cancer Res. 2004; 64(20):17237-7236; Fukino K, et al. JAMA. 2007; 297(19):2103-2111) Briefly, DNA from germline and tumor was amplified using 1 of 72 multiplex primer panels of fluorescent-labeled microsatellite markers (MapPairs genome-wide Human Markers set versions 9 and 10; Invitrogen, Carlsbad, Calif.). A total of 345 autosomal markers were common to all samples and were used for the analysis. Genotyping was performed with the AB13700 or 3730×1 semiautomated sequencer (Applied Biosystems, Perkin-Elmer Corp, Norwalk, Conn.). The results were analyzed by automated fluorescence detection using GeneScan collection and analysis software (Applied Biosystems).

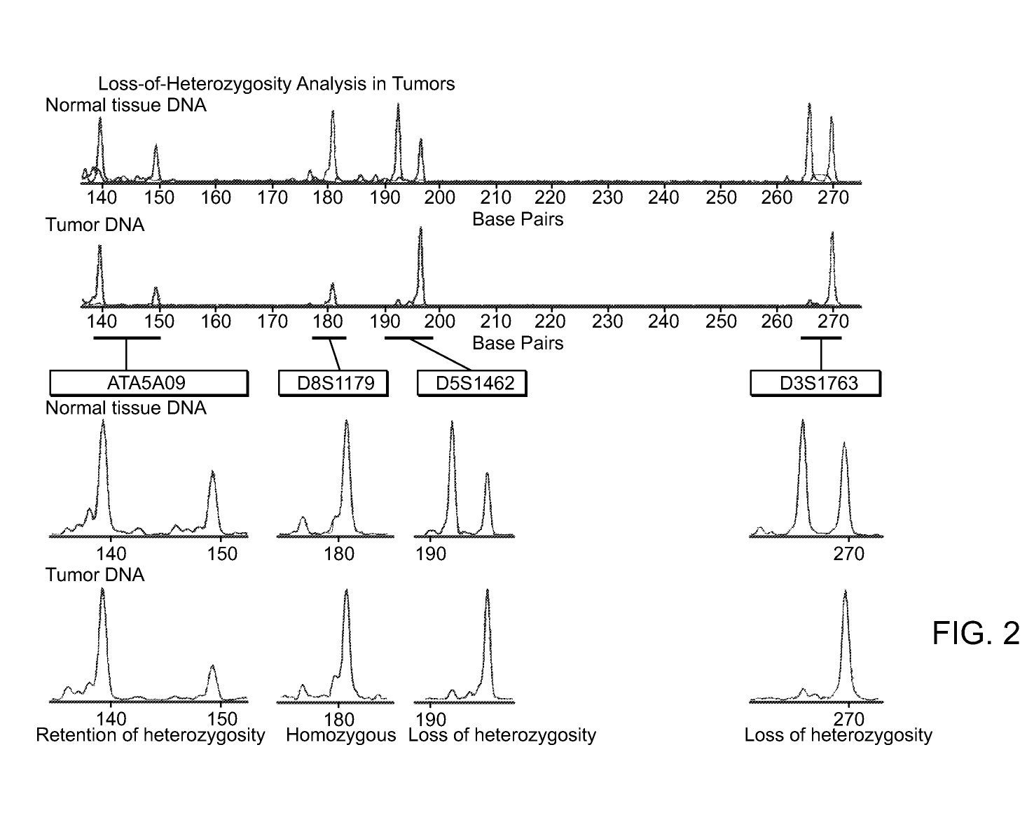

Each marker was analyzed in genomic DNA from the germline and tumor from each patient. The germline could be scored as homozygous (single peak in the normal tissue) (FIG. 2), heterozygous (2 peaks) (FIG. 2), or failure (failed polymerase chain reaction). For each heterozygous marker in the germline, somatic LOH/AI in the corresponding tumor (FIG. 2) was scored as present when the ratio of peak heights of alleles between germline and tumor DNA was more than 1.5 or less than 0.66, and retention of heterozygosity (FIG. 2) was noted when the ratio of peak heights was less than 1.5. A reaction failure was noted when the amplification reaction had failed. In most cases, the ratio of ratios was 1.8 to 2.2 or 0.5 for LOH/AI.

### Statistical Analysis of Microsatellite Genotyping

All statistical analyses were performed using R version 2.4. (R Development Core Team, R: a Language and Environment For Statistical Computing. Vienna, Austria: University of Vienna; 2007) A total of 345 autosomal microsatellite markers were analyzed in germline and in tumor DNA samples from 385 patients. For each marker, the frequency of germline homozygosity was determined as the number of homozygous markers divided by the sum of heterozygous plus homozygous markers. In addition, for each marker, the frequency of LOH/AI was determined as the number of LOH/AI events divided by the sum of the LOH/AI and retention-of-heterozygosity events. For each marker, a median of 105 samples failed either in germline or in tumor because of polymerase chain reaction failures.

It was observed that missing information was more abundant for markers with decreased frequency of germline homozygosity and with increased frequency of LOH in tumor. In statistical terminology, this is known as nonignorable inissingness and therefore must be addressed. Toward this end, a statistical procedure to estimate the heterozygosity and LOH frequencies in the observed and unobserved (due to genotype failure) data combined was designed. That is, the true frequencies for all microsatellites were estimated as follows. First, initial rough estimates of genotype failure rates were made separately for homozygote, heterozygote, lost (LOH), and retained loci. Such estimates automatically imply, via standard conditional probability, heterozygosity and LOH frequencies for each microsatellite. These individual microsatellite estimates in turn can be used to provide more accurate estimates of the genotype failure rates. This procedure is iterated repeatedly until all estimates converge to optimal solutions (mathematical details available at http://www lerner.ccf.org/gmi/igac). In cancer type-specific investigations such as this, and to minimize ascertainment bias, germline homozygosity frequencies were not determined for markers with fewer than 5 informative patients (2 markers in patients with prostate cancer). Similarly, LOH/AI frequencies for any markers with fewer than 5 informative tumors (1 marker in head and neck squamous cell carcinomas and 4 markers in the prostate cancers, including the 2 germane failures) were not calculated. Five (5) were chosen as a reasonable cutoff under which computing a frequency is not reliable from our experience.

For each marker, the frequency of germline homozygosity was compared with the binomial distribution using a reference germline homozygosity frequency as the null homozygosity probability. The number of homozygotes for a marker was determined as the product of the corrected germline homozygosity frequency for this marker and the number of informative patients. The reference frequencies for a western European ancestry population were provided for each marker by the Cooperative Human Linkage Center (Marshfield, Wis.). These are ancestry-matched controls because our cases are white individuals of western European ancestry. Similarly, LOH/AI frequencies were compared for each marker with the binomial distribution using the average LOH/AI frequencies of the markers on the same chromosome as the null LOH/AI probability. The number of LOH/AI events for a marker was determined as detailed earlier. For testing for both germline homozygosity frequencies and LOH/AI frequencies, a 1-sided exact binomial test was used (R function pbinom, FALSE), and P values were adjusted for multiple-testing false discovery rate over the 345 markers (R function p.adjust, method=“FDR”). Adjusted P values less than 0.05 were considered significant. The overall relationship between germline homozygosity frequency and somatic LOH/AI frequency across all markers was measured by the Pearson correlation test (R function cor.test).

Validation with Single-Nucleotide Polymorphism Microarray Data

A publicly available data set of paired germline and somatic DNA from 205 non-small cell lung carcinomas, which had been subjected to genome-wide single-nucleotide polymorphism (SNP) genotyping with the 250K SNP Chip (Affymetrix, Santa Clara, Calif.) (Weir B A, et al., Nature, 2007; 450(7171):893-898), was used to validate the microsatellite data. All 205 samples came from individuals of European ancestry, confirmed with their SNP genotypes in comparison with the HapMap 3 populations using the program Structure (http://pritch.bsd.uchicago.edu/software.html). Both cases and controls were of similar SNP-based ancestry. For each patient, both a tumor sample (fresh/flash frozen) and a normal tissue sample (peripheral blood leukocytes and fresh/flash frozen normal lung tissue) were available. This data set did not have nonignorable missingness. Loss of heterozygosity was identified using dChipSNP (Lin M, et al. Bioinformatics 2004:20(8)1233-1240). dChipSNP was applied to the tumor and germline genotypes using the paired analysis setting. All other parameters were set to default values.

For each SNP, the frequency of homozygosity in the germline was obtained by dividing the number of homozygous genotypes, AA and BB, by the total number of genotypes, AA, AB, and BB (i.e., homozygous and heterozygous genotypes), in the germline. The frequency of homozygosity was considered for the SNPs within 100 kilobase (kb) of the 12 microsatellite markers shown to have increased homozygosity in cancer patients (the remaining 4 regions could not be assessed because the relationship of SNPs to these markers remain imprecisely mapped). The frequency of homozygosity in the germ-line of patients with lung cancer was compared with that of HapMap controls of European ancestry using the distribution function of the binomial distribution, considering 1-sided P values (R function pbinom). These exact binomial test P values were adjusted by false discovery rate (R function FDR).

For each SNP, the frequency of LOH in the lung cancer samples was determined as the proportion of SNPs with somatic LOH. Correlations between the frequency of homozygosity in the germ-line and the frequency of LOH in tumors at each SNP were performed using the Pearson correlation test (cor.test in R). For that correlation, frequency of germline homozygosity was determined among SNPs that did not show LOH in tumors, and the frequency of LOH in tumors was determined among SNPs that were germline heterozygous. To take into consideration the low frequency of LOH when using SNPs (mean, 1.7%/SNP), frequencies were computed only for SNPs with at least 100 informative samples.

### Results

Germline homozygosity was explored using 345 autosomal microsatellite markers in 385 patients with 3 different types of invasive carcinomas. These markers were selected for their high heterozygosity index and their regular spacing throughout the genome (Weber L. et al. Am. J. Hum. Genet. 1989; 44 (3):388.396; Rosenberg N A, et al. Science 2002: 298(5602):2381-2385). In the first instance, a frequency of germline homozygosity was determined at each marker (or locus) in the overall (merged) set comprising all 3 groups of patients who had breast, prostate, or head and neck squamous cell carcinomas. This yielded 114 loci with increased frequencies of germline homozygosity compared with those of ancestry-matched controls (adjusted P values <0.05 to <0.001).

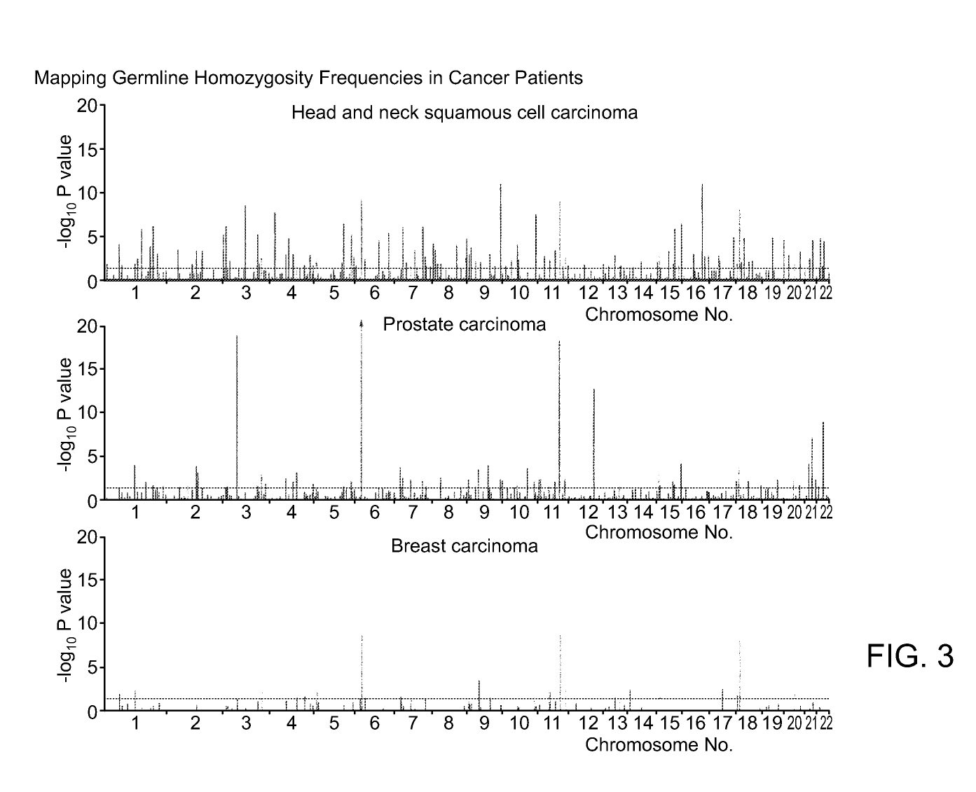

Each group of patients with each cancer type was then studied (FIG. 3). 83 loci in head and neck squamous cell carcinoma, 56 loci in prostate cancer cases, and 26 loci in breast cancer cases with statistically significantly higher frequencies of germ-line homozygosity compared with those of ancestry-matched controls in each group of patients (adjusted P values <0.05 to <0.001) were identified (FIG. 3).

Among the 345 loci studied, 120 loci had higher-than-predicted frequencies of homozygosity in patients with head and neck squamous cell carcinoma, 69 loci in patients with prostate cancer, and 27 loci in patients with breast cancer (FIG. 3). Sixteen of these loci were in common among the 3 groups of patients with the 3 cancer types (Table 1). Copy number variations are numerous throughout the genome (Radon R, et al. Nature 2006; 444(7118):444-454) and can potentially generate chromosomal regions with a single copy of DNA (hemizygous). To explore whether these 16 loci could be hemizygous due to copy number variations, the Database for Genomic Variants (http://projects.tcag.ca/variation/project.html) was screened to identify whether any of these 16 loci identified to have high germline homozygosity frequencies lie within known regions with copy number variations. Only 1 of the 16 loci, D11S1993, is included in such a copy number variation region. As a comparison, among the entire genome-wide set of 345 markers used in this study, 20 are in copy number variation regions, which is proportionally similar (binomial test; P=0.70). In summary, these data show that germline homozygosity at specific markers is more frequent in cancer patients.

The frequency of somatic events occurring at these 16 loci was then analyzed by looking at the frequency of somatic LOH/AI in heterozygote patients. The mean (SD) LOH/AI frequency at the 16 loci in the 3 cancer types was 58% (4.2%) (range, 48%-65%). As a comparison, we selected 197 microsatellite markers that were not found to have an increased frequency of germline homozygosity in patients with any of the 3 cancer types compared with that of controls. The mean (SD) LOH/A1 frequencies at these 197 loci was 50% (7.5%) (range, 34%-70%), which is statistically significantly lower than the mean 58% LOH/AI frequency at the 16 loci (P<0.001).

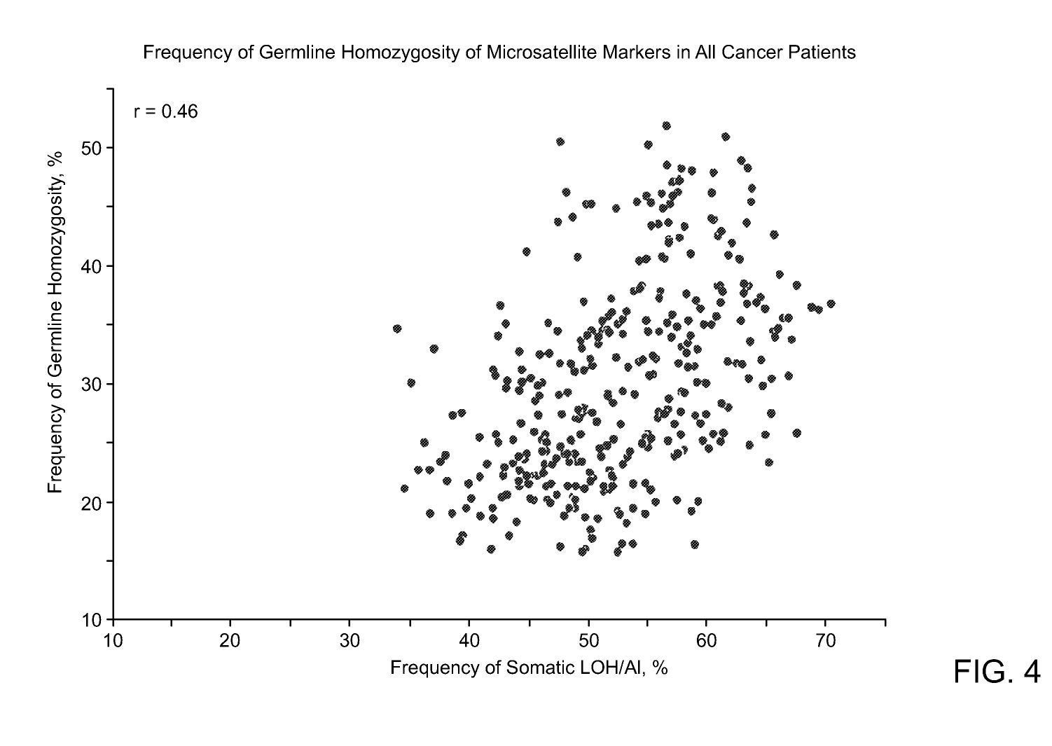

Thus, it has been shown that if cancer patients are heterozygous at these 16 foci that have an increased germline homozygosity, then their corresponding tumors have a higher frequency of somatic-LOH/AI at these same 16 loci. In light of this observation, whether this phenomenon could be generalizable was explored. The frequencies of homozygosity or heterozygosity among all markers is linear (and not binary). It was hypothesized that if the first observation with the 16 loci was generalizable, then it would also be observed that as loci have increasing frequencies of germ-line homozygosity, those same loci would also have increasing somatic LOH/AI frequencies in the tumors of cancer patients who happened to be heterozygous at these loci. Indeed, it was found that this observation could be extended to all 345 microsatellite markers, where a correlation between the frequencies of germline homozygosity and the frequencies of somatic LOH/AI in patients who happened to be heterozygous at the same loci were identified (r=0.46, 95% confidence interval, 0.37-0.53, P<0.001) (FIG. 4).

Thus far, it has been shown that for loci with increased germline homozygosity among all cancer patients, these same loci that happened to be germ-line heterozygous in some cancer patients also have an increased LOH/AI frequency in their corresponding tumors (FIG. 4). Furthermore, it has been shown that this observation can be generalizable; i.e., the more frequent the homozygosity at any given locus, the more LOH/AI can be found at those loci in the tumors of cancer patients that happened to be heterozygous in the germ-line (FIG. 4).

To further corroborate this generalized association, the loci with the highest frequency of somatic LOH/A1 events across all the 3 types of cancers was first determined. After identifying these “hot-spot” LOH/AI loci, whether these loci also exhibited (relatively) high frequencies of homozygosity across the germlines of cancer patients was then determined. First, loci with high LOH/AI frequencies were identified for each chromosome. Then a list of markers was tabulated that represented those with a significant elevation of both germline homozygosity and LOH/AI frequencies for the entire cohort and for each specific cancer type (Table 2). A total of 22 hot spots were identified, including 21 common to the entire series, 1 in the head and neck squamous cell carcinoma group, 4 in the prostate cancer group, and 0 in the breast cancer group. All 22 of the “overall” hot-spot loci (Table 2) are also found among the regions initially identified as having increased homozygosity frequencies. Again, only 1 of these 22 hot spots (D1 6S2621) is included in a copy number variation region.

To independently validate these observations, an existing published data set comprising SNP genotypes of paired germline and tumor DNA from 205 white patients with lung carcinoma was used. This publicly available data set provided a different solid tumor, which happens to be the most common solid tumor in men, assessed with a different genotyping technique. The frequencies of homozygosity were compared with HapMap data, and at least 1 SNP (range, 1-16) in each of the 11 of the 12 previously noted evaluable “highly homozygous” regions was found to be significantly more homozygous in lung cancer cases compared with controls (P<0.05 to P<0.001) (eTable, available online at http://www.jama.com). Thus, the observations of increased germline homozygosity at specific markers in the original 3 solid tumor types were independently replicated. The lung cancer SNP data set was then used to validate the previously noted association between frequency of germline homozygosity and that of somatic LOH. Confirming the data in breast, prostate, and head and neck carcinomas, a direct correlation between LOH frequencies and those of germline homozygosity at the corresponding SNPs in the germline that happens to be heterozygous was found (r=0.21, 95% confidence interval; 0.18-0.24, P<0.001).

The data provided herein indicate that there are specific common loci that are prone to homozygosity in the germline of individuals with breast, prostate, and head and neck squamous cell carcinomas. Importantly, the observations were independently validated in a different type of solid tumor, lung carcinoma, by showing an increased frequency of germline homozygosity in cancer cases compared with ancestry-matched controls. It is of interest to note that the most frequently homozygous SNPs were, for the most part, in proximity to the original microsatellites found to be “most homozygous” in the other 3 solid tumors (eTable). The correlation between germline homozygosity and increased somatic LOH at the same marker when the latter's germline happens to be heterozygous was also confirmed.

There are at least 2 hypotheses to explain the increased germline homozygosity in cancer cases compared with controls. First, random mating would generate random homozygosity, and when this homozygosity occurs at enough loci harboring genes relevant to cancer with pathogenic alleles, cancer risk is increased. This could explain the increased homozygosity at specific loci observed in cancer patients compared to controls. If this were true, then it would be expected that homozygosity would favor the frequent alleles. However, this cannot explain the high somatic LOH/AI frequencies observed at these same loci when the germline happens to be heterozygous. Therefore, an alternative hypothesis has to be considered.

Increased germline homozygosity may be postulated to occur at common fragile sites, which are specific loci with increased sensitivity to DNA damage (Hueoner K, et al., Nat Rev Cancer 2001; 1(3):214-221). Common fragile sites, which are scattered across the genome, should be distinguished from “rare” fragile sites, which are also sensitive to DNA damage, because the latter harbor expanded tandem nucleotide repeats that common sites do not have. Hence, the common fragile sites would be good candidate loci to have high frequencies of germline homozygosity in cancer patients and high LOH/AI frequencies in tumors, because they are prone to both homologous recombination as a mechanism of DNA repair after double-strand breaks (Jackson S P., Carcinogenesis 2002; 23(5):687-696) and to rearrangements in tumors, including L0H/AI (Hueoner K, et al., Nat Rev Cancer 2001; 1(3):214-221)

Remarkably, 1 hot-spot marker we identified (D8S1128) is approximately 250 kb from a common fragile site (MYC). Two additional hot spots (D16S2621 and GATA178F11) are 250 kb from 2 rare fragile sites (HDL2 and TYMS) related to triplet nucleotide repeats. However, for these rare fragile sites, the link with somatic LOH/AI is not well established. Nonetheless, the data described herein contributes to this link given that the data derive from microsatellite markers, which comprise nucleotide repeats of various repeat lengths. It would, therefore, be rather interesting to distinguish the precise mechanisms of the association of germline homozygosity and cancer susceptibility as well as its link to increased LOH when the germline happens to be heterozygous.

Some may be surprised that the markers associated with traditional high-penetrance susceptibility genes are not prominent among our highly homozygous list. Currently, there are no high-penetrance susceptibility genes for prostate, lung, or head and neck carcinomas. If the group of patients with breast cancers is focused upon, the regions associated with high-penetrance inherited breast cancer syndromes, namely, BRCA1 (17q12), BRCA2 (13q14), PTEN (10q23.3), or TP53 (17p13-p15) (Table 1) were not seen. This non-association may contribute to the overall hypothesis that regions of germline homozygosity could represent low-penetrance factors predisposing to carcinoma, at least to these 3 cancer types. In contrast, there are at least 3 SNPs identified and replicated as associated with breast cancer cases (Cox A., et al., Nat. Genet. 2007; 39(3):352-358; Easton D F, et al. Nature 2007; 447(7148):1087-1093) that are also found in our regions of high germline homozygosity in our patients with breast cancer, namely, 10q26 (SNP in proximity to FGFR2), 5q1-3 (MAP3K1), and 11p15.5 (LSP1).

Similarly, SNPs representing several different loci in the 8q24 region have been shown and validated to be associated with prostate cancer cases (Yeager M, et al. Nat. Genet. 2007; 39(5):645-649; Hunter D J, et al. Nat. Genet. 2007:39(7):870-874) and this 8q24 region is also well represented among the loci with high frequencies of germline homozygosity in the prostate cancer cases. Interestingly, and consistent with the present observations; this 8q24 region also harbors SNPs associated with colorectal and other cancers (Tomlinson I, et al. Nat. Genet. 2007:39(8):984-988; Zanke B W, et al. Nat. Genet. 2007, 39(8):989-994), indicating a more general cancer susceptibility allele or alleles in this region. The current data reveal several distinct markers in 8q24 with high frequencies of germline homozygosity and LOH for each tumor group as well as in the overall merged series of all patients with these breast, prostate, and head and neck cancers (Table 2).

Cancer susceptibility loci are currently sought by association studies, whereas somatic alterations are most often identified separately. The recent availability of high-density arrays considerably increased the resolution of these studies. (Cox A., et al., Nat. Genet. 2007; 39(3):352-358; Easton D F, et al. Nature 2007; 447(7148):1087-1093; Hunter D J, et al. Nat. Genet. 2007:39(7):870-874; Tomlinson I, et al. Nat. Genet. 2007:39(8):984-988; Zanke B W, et al. Nat. Genet. 2007, 39(8):989-994; Gudmundsson J. et al. Nat. Genet. 2007:39151:631-637). However, currently, somatic and germline cancer genetics are 2 separate fields. In fact, from germline inheritance of a predisposing allele to cancer development is still somewhat of a mechanistic “black box”. The observations described herein link the somatic and germline cancer genetic fields. These observations are also consistent with cancer as a complex genetic trait if one considers that carcinogenesis occurs when a minimal number of somatic events are required for a cell to become malignant. Continuing this concept, therefore, germline homozygosity at specific loci represents a low penetrance but common etiology for cancer and serves as a parallel but equivalent pathway to accumulation of somatic LOH.

### CONCLUSIONS

The data described herein derived from 3 different solid tumors, validated in a fourth, demonstrate that high frequencies of germline homozygosity at specific markers are associated with these cancers compared with controls and that at the genome-wide level, there is a direct relationship between frequencies of germline homozygosity and somatic LOH if the germline is heterozygous. Although the total number of cancer cases nears 600, the sample size per solid tumor type is still relatively small (116-205). Therefore, the observations here can be further validated in these solid tumors and explored in other malignancies. Robustly replicating the data independently, further validates that germline homozygosity at specific loci as low-penetrance alleles predisposing to carcinomas be taken into account in cancer risk assessments and management beyond high-penetrance cancer susceptibility genes. Additionally, with further studies and fine structure analyses, it is likely such data predicts the likelihood of LOH in a tumor at specific genomic loci if the relative frequencies of germline homozygosity/heterozygosity at those same loci are known.

TABLE 1
Sixteen Microsatellite Markers With Elevated Frequencies of Germline
Homozygosity Common to 3 Cancer Types^{a}

| | | P Value | | | |
|---|---|---|---|---|---|
| | | | Prostate | Breast | |
| Marker | Locus | HNSCC | Cancer | Cancer | Genes Within 250 kb |
| D2S1790 | 2p11.2 | <.01 | <.05 | <.001 | TMSB10, KCMF1, TCF7L1 |
| D3S2427 | 3q26.31 | <.001 | <.01 | <.01 | |
| D4S2394 | 4q28.2 | <.001 | <.001 | <.05 | |
| D5S2505 | 5p15.32 | <.001 | <.05 | <.01 | |
| D6S1959 | 6p22.3 | <.01 | <.001 | <.001 | |
| D7S3046 | 7q11.22 | <.001 | <.01 | <.05 | DFNB39 |
| D9S1871 | 9p24.2 | <.01 | <.01 | <.05 | GLIS3 |
| D10S1222 | 10q26.2 | <.01 | <.01 | <.05 | DOCK1, FGFR2 |
| D11S1993 | 11p11.2 | <.01 | <.05 | <.01 | API5, TTC17, HSD17B12 |
| D11S1986 | 11q23.2 | <.001 | <.001 | <.001 | POU2AF1, BTG4, LAYN |
| D11S4463 | 11q24.3 | <.05 | <.01 | <.01 | BARX2 |
| D13S793 | 13q31 | <.001 | <.05 | <.05 | |
| D15S822 | 15q12 | <.001 | <.001 | <.01 | GABRA5 |
| GATA178F11 | 18p11.31 | <.001 | <.01 | <.05 | |
| D18S1376 | 18p11.31 | <.001 | <.001 | <.001 | ZFPI61, EPB41L3 |
| D20S477 | 20p11.21 | <.001 | <.01 | <.05 | FOXA2 |

Abbreviations:
HNSCC, head and neck squamous cell carcinoma;
kb, kilobase.
^{a}Adjusted P values for all 3 solid tumor sets combined at these 16 markers <.001.
All comparisons were with controls.

TABLE 2
Germline Homozygosity and Somatic LOH/AI Hot Spots^{a}

| | | P Value | | | | | | | | |
|---|---|---|---|---|---|---|---|---|---|---|
| | | All | | HNSCC | | Prostate | | Breast Cancer | | |
| | | Homo- | | Homo- | | Homo- | | Homo- | | |
| Marker | Locus | zygous^{b} | LOH^{b} | zygous^{b} | LOH^{b} | zygous^{b} | LOH^{b} | zygous^{b} | LOH^{b} | Genes at or Within 250 kb |
| D1S2134 | 1p33 | <.001 | <.01 | <.05 | .61 | .14 | <.05 | .31 | .30 | |
| D1S1589 | 1q25.1 | <.01 | <.05 | .40 | .60 | <.01 | .44 | >.99 | .38 | RABGAP1L, GPR52 |
| D1S3462 | 1q42.2 | <.001 | <.05 | <.01 | .85 | <.05 | .22 | >.99 | .33 | DISC1 DISC2, TSNAX |
| D2S1334 | 2q21.3 | <.001 | <.05 | <.001 | .46 | <.001 | <.01 | .28 | .68 | R3HDM1, MCM6, LCT, |
| | | | | | | | | | | ZRANB3, DARS, |
| | | | | | | | | | | UBXD2 |
| D2S1776 | 2q24.3 | <.001 | <.05 | <.001 | .61 | <.05 | .11 | >.99 | .37 | LASS6, ABCB11, |
| | | | | | | | | | | G6PC2, SPBC25, |
| | | | | | | | | | | NOSTRIN |
| D3S2409 | 3p21.31 | <.001 | <.05 | .63 | .05 | <.001 | .30 | .06 | .75 | RHOA, GPX1, USP4, |
| | | | | | | | | | | AMT, CCDC71, LAMB2, |
| | | | | | | | | | | USP19, NICN1, DAG1, |
| | | | | | | | | | | CCDC36 |
| D3S1746 | 3q25.1 | <.001 | .14 | <.001 | .90 | .<05 | <.05 | .09 | .56 | SUCNR1, AADAC, |
| | | | | | | | | | | MBNL1 |
| D3S1763 | 3q26.1 | <.001 | <.05 | <.05 | .81 | .12 | <.05 | >.99 | .44 | WDR49, PDCD10, |
| | | | | | | | | | | SERPIN1 |
| D3S2427 | 3q26.3 | <.001 | <.05 | <.001 | .61 | <.01 | <.01 | <.01 | .87 | |
| D4S2623 | 4q25 | <.001 | <.01 | <.01 | .46 | <.01 | .38 | >.99 | .06 | EGF, ELOVL6, CFI, |
| | | | | | | | | | | NOLA1, PLA2G12A, |
| | | | | | | | | | | RRH |
| D8S1128 | 8q24.21 | <.05 | <.01 | <.05 | .32 | <.05 | .37 | >.99 | .27 | MYC, PVT1 |
| D9S1121 | 9p21.3 | <.01 | <.05 | <.01 | .67 | <.05 | .27 | >.99 | <.05 | TUSC1 |
| D10S1423 | 10p12.33 | <.05 | <.05 | <.01 | .32 | .22 | .27 | >.99 | .38 | C10orf112 |
| D11S1986 | 11q23.2 | <.001 | <.05 | <.001 | .52 | <.001 | .34 | <.001 | .30 | POU2AF1, BTG4, LAYN |
| D11S4464 | 11q24.1 | <.05 | <.05 | <.05 | .46 | .21 | .19 | >.99 | .38 | OR6X1, ZNF202, |
| | | | | | | | | | | SCN3B, |
| | | | | | | | | | | OR6M1, PMP22, |
| | | | | | | | | | | OR8D4 |
| D12S2078 | 12q24.32 | <.01 | <.001 | .55 | .05 | .35 | <.05 | .96 | .61 | |
| D14S1280 | 14q12 | <.05 | <.05 | <.05 | .42 | .09 | .64 | >.99 | .30 | NOVA1 |
| D15S655 | 15q25.3 | <.05 | <.05 | <.05 | .26 | <.01 | .81 | >.99 | .30 | TMEM83 |
| D16S422 | 16q23.3 | <.05 | <.01 | <.01 | .05 | >.99 | .22 | .96 | .46 | CDH13 |
| D16S2621 | 16q24.2 | <.01 | <.05 | <.01 | .96 | .14 | <.05 | >.99 | .30 | JPH3, KLHDC4, |
| | | | | | | | | | | SLC7A5, CA5A, BANP |
| D21S2055 | 21q22.2 | <.001 | <.05 | <.001 | <.05 | <.001 | .28 | .14 | .75 | PCP4, DSCAM, |
| | | | | | | | | | | B3GALT5 |
| D22S683 | 22q12.3 | <.001 | <.001 | <.001 | .05 | <.001 | <.01 | >.99 | .39 | APOL1 through APOL4, |
| | | | | | | | | | | MYH9 |

Legend for Table 2:
Abbreviations:
HNSCC, head and neck squamous cell carcinoma;
kb, kilobase;
LOH/AI, loss of heterozygosity/allelic imbalance.
^{a}Note adjusted P values <.05 are considered significantly elevated homozygosity frequencies or somatic LOH/AI frequencies. Comparisons of germline homozygosity were against controls. Comparisons of LOH frequencies were against an internal mean for each tumor type.
^{b}Adjusted P values of significantly elevated germline homozygosity frequency and somatic LOH/AI frequency are provided across the whole data set (All) and for individual cancer types.

### Legend for Table 2:

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of diagnosing cancer in an individual in need thereof comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity at the one or more loci is indicative of a diagnosis of cancer in the individual.

2. The method of claim 1 wherein the homozygosity at the one or more loci occurs in the individual's germline nucleic acid, somatic nucleic acid, or a combination thereof.

3. The method of claim 1 wherein the cancer is breast cancer, prostate cancer, head and neck squamous cell carcinoma, lung cancer, or a combination thereof.

4. The method of claim 1 further comprising obtaining a sample comprising the individual's nucleic acid from the individual.

5. The method of claim 4 wherein the sample is a blood sample, a cell sample, a tissue sample, or a combination thereof.

6. The method of claim 5 wherein the sample is obtained from normal tissue, cancerous tissue, or a combination thereof.

7. The method of claim 6 wherein the normal tissue is peripheral blood leukocytes, lung tissue, or a combination thereof.

8. The method of claim 6 wherein the cancerous tissue is tissue from a tumor.

9. The method of claim 8 wherein the cancerous tissue obtained from the tumor is carcinomatous epithelium of the tumor.

10. The method of claim 1 wherein the homozygosity at the one or more loci of the individual's nucleic acid is detected using polyermase chain reaction to amplify the individual's nucleic acid thereby producing amplified nucleic acid, and genotyping the amplified nucleic acid.

11. The method of claim 1 wherein the homozygosity at the one or more loci of the individual's nucleic acid is compared to the homozygosity at the one or more loci of a control, and if frequency of the homozygosity at the one or more loci of the individual's nucleic acid is increased compared to frequency of the homozygosity at the one or more loci of the control, then cancer is diagnosed in the individual.

12. The method of claim 1 wherein the individual is a human.

13. A method of diagnosing susceptibility to cancer in an individual in need thereof comprising detecting homozygosity at one or more loci of the individual's nucleic acid, wherein the one or more loci is selected from the group consisting of: D2S1790, D3S2427, D4S2394, D5S2505, D6S1959, D7S3046, D9S1871, D10S1222, D11S1993, D11S1986, D11S4463, D13S793, D15S822, GATA178F11, D18S1376, and D20S477, and homozygosity at the one or more loci is indicative of a diagnosis of susceptibility to cancer in the individual.

14. The method of claim 13 wherein the homozygosity at the one or more loci occurs in the individual's germline nucleic acid, somatic nucleic acid, or a combination thereof.

15. The method of claim 13 wherein the cancer is breast cancer, prostate cancer, head and neck squamous cell carcinoma, lung cancer, or a combination thereof.

16. The method of claim 13 further comprising obtaining a sample comprising the individual's nucleic acid from the individual.

17. The method of claim 16 wherein the sample is a blood sample, a cell sample, a tissue sample, or a combination thereof.

18. The method of claim 17 wherein the sample is obtained from normal tissue, cancerous tissue, or a combination thereof.

19. The method of claim 18 wherein the normal tissue is peripheral blood leukocytes, lung tissue, or a combination thereof.

20. The method of claim 18 wherein the cancerous is tissue obtained from a tumor.

21. The method of claim 20 wherein the cancerous tissue obtained from the tumor is carcinomatous epithelium of the tumor.

22. The method of claim 13 wherein the homozygosity at the one or more loci of the individual's nucleic acid is detected using polyermase chain reaction to amplify the individual's nucleic acid thereby producing amplified nucleic acid, and genotyping the amplified nucleic acid.

23. The method of claim 13 wherein the homozygosity at the one or more loci of the individual's nucleic acid is compared to the homozygosity at the one or more loci of a control, and if frequency of the homozygosity at the one or more loci of the individual's nucleic acid is increased compared to frequency of the homozygosity at the one or more loci of the control, then the individual is susceptible to cancer.

24. The method of claim 13 wherein the individual is a human.
